(19) United States
(12) Patent Application Publication
(10) Pub. No.: US 2026/0186532 A1
(43) Pub. Date: Jul. 2, 2026

(54) ELECTRONIC DEVICE INCLUDING MULTIPLE SENSORS

(71) Applicant: SAMSUNG DISPLAY CO., LTD., YONGIN-SI (KR)

(72) Inventor: Won Sang PARK, Yongin-si (KR)

(21) Appl. No.: 19/377,336

(22) Filed: Nov. 3, 2025

(30) Foreign Application Priority Data
Dec. 31, 2024 (KR) 10-2024-0202590

(51) Int. Cl. G06F 1/16; G06F 1/18; G06F 3/01

(52) U.S. Cl. CPC G06F 1/163; G06F 1/1658; G06F 1/1694; G06F 1/1698; G06F 1/183; G06F 3/014

(57) ABSTRACT

An electronic device including a substrate, a first sensor assembly, a second sensor assembly, and a signal processor. The first sensor assembly is disposed on the substrate. The first sensor assembly includes a light emitter and a first optical sensor adjacent to the light emitter, and is configured to output a first detection signal. The second sensor assembly includes a second optical sensor spaced apart from the first sensor assembly and disposed on the substrate, and is configured to output a second detection signal. A distance between the second optical sensor and the light emitter is greater than a distance between the second optical sensor and the first optical sensor. The signal processor is configured to receive the first detection signal and the second detection signal, and to generate a refined detection signal based on the first detection signal and the second detection signal.

### CROSS-REFERENCE TO RELATED APPLICATION

This non-provisional patent application claims priority under 35 U.S.C. § 119 to Korean Patent Application No. 10-2024-0202590 filed on Dec. 31, 2024 in the Korean Intellectual Property Office, the disclosure of which is incorporated by reference herein in its entirety.

### FIELD

The present disclosure relates to an electronic device, and more particularly, to an electronic device including multiple sensors.

### DISCUSSION OF THE RELATED ART

With the development of electronic technology, the use of various portable electronic devices such as smartphones, tablet PCs, laptop PCs, and personal digital assistants (PDAs) is increasing.

To enhance the portability electronic devices, wearable electronic devices to be worn on the body are gaining widespread demand, and research on the direction of the application of these wearable electronic devices is conducted.

The wearable electronic devices are structures that may be worn on the body or parts of the human body, and various types of products are being developed due to the merits of being convenient to wear and allowing the user to use both hands freely.

Recently, the wearable electronic devices have been increasingly utilized for applications such as personal authentication, healthcare, etc. by leveraging direct contact with parts of the body. To this end, active research is conducted on detection devices for detecting biometric information such as blood flow and pulse that may be included in the wearable electronic devices.

### SUMMARY

Embodiments are intended to provide an electronic device with enhanced accuracy of a signal measurement using light. Embodiments are intended to provide a wearable electronic device with enhanced accuracy of measuring biological signals.

An electronic device including a substrate, a first sensor assembly, a second sensor assembly, and a signal processor. The first sensor assembly may be disposed on the substrate. The first sensor assembly may include a light emitter and a first optical sensor adjacent to the light emitter, and the first sensor assembly may be configured to output a first detection signal. The second sensor assembly may include a second optical sensor spaced apart from the first sensor assembly and disposed on the substrate, and a distance between the second optical sensor and the light emitter may be greater than a distance between the second optical sensor and the first optical sensor, and the second sensor assembly may be configured to output a second detection signal. The signal processor may be configured to receive the first detection signal and the second detection signal, and to generate a refined detection signal based on the first detection signal and the second detection signal.

The electronic device may further include a first conductive line disposed on the substrate. The first conductive line may connect the first sensor assembly and the signal processor, and the first conductive line may connect the second sensor assembly and the signal processor. The electronic device may further include a first insulation layer disposed on the first conductive line, and the first insulation layer may include a connection hole. The electronic device may further include a first electrode that overlaps the connection hole and may connect the first sensor assembly and the first conductive line, and may connect the second sensor assembly and the first conductive line.

The first optical sensor may completely surround the light emitter. The first optical sensor partially surrounds the light emitter.

The electronic device may include a first insulation layer disposed on the substrate, and a second conductive line disposed on the first insulation layer and connecting the first sensor assembly and the signal processor, and connecting the second sensor assembly and the signal processor.

The first optical sensor may include a first part and a second part, the first part and the second part may be disposed on opposite sides of the light emitter, and may be spaced apart from each other. The second optical sensor may include a third part and a fourth part, the first part may be disposed between the light emitter and the third part, and the second part may be disposed between the light emitter and the fourth part.

The electronic device may further include a first conductive line disposed on the substrate and connecting the first sensor assembly and the signal processor, a first insulation layer including a connection hole disposed on the first conductive line, a first electrode overlapping the connection hole, and a second conductive line disposed on the first insulation layer and connecting the second sensor assembly and the signal processor.

The electronic device may further include a second insulation layer disposed on the substrate, the second insulation layer may include a first opening, a second opening, and a third opening, and the first optical sensor may overlap the first opening, the light emitter may overlap the second opening, and the second optical sensor may overlap the third opening.

The second insulation layer may include a light blocking material. The light emitter may include an organic light emitting diode.

The light emitter may include a first sub-light emitter configured to emit a light having a first wavelength and a second sub-light emitter configured to emit a light having a second wavelength different from the first wavelength.

The electronic device may further include a second electrode covering the light emitter, the first optical sensor, and the second optical sensor.

A wearable electronic device including a housing that includes an inner cover defining an inner surface of a wearable electronic device, an electronic part disposed in the housing, and a signal processor configured to receive a first detection signal and a second detection signal, and to generate a refined detection signal based on the first detection signal and the second detection signal. The electronic part includes a substrate, an optical sensor component including a first sensor assembly and a second sensor assembly disposed on the substrate. The first sensor assembly includes a light emitter and a first optical sensor adjacent to the light emitter, and configured to output a first detection signal. The second sensor assembly includes a second optical sensor spaced apart from the first sensor assembly, and configured to output a second detection signal. A distance between the second optical sensor and the light emitter is greater than a distance between the second optical sensor and the first optical sensor.

The light emitter may emit light toward the inner surface.

A first end and a second end of the optical sensor component may be connected to a first end and a second end of the signal processor, respectively.

The optical sensor component may include a plurality of first sensor assembly and a plurality of second sensor assembly, each of the plurality of first sensor assembly and each of the plurality of second sensor assembly may be alternately arranged.

The housing may have a ring shape.

An electronic device including a first sensor assembly that includes a light emitter and a first optical sensor disposed adjacent to the light emitter, the light emitter may be configured to emit light and the first sensor assembly may be configured to output a first detection signal corresponding to the light reflected from an object and ambient light. The electronic device may include a second sensor assembly that includes a second optical sensor spaced apart from the first sensor assembly, the second sensor assembly may be configured to output a second detection signal corresponding to the ambient light reflected from the object. The electronic device may include a signal processor configured to receive the first detection signal and the second detection signal, and to generate a refined detection signal based on the first detection signal and the second detection signal.

The electronic device may include a first conductive line connecting the first sensor assembly and the signal processor, and connecting the second sensor assembly and the signal processor, a first insulation layer disposed on the first conductive line, the first insulation layer may include a connection hole, and a first electrode that overlaps the connection hole and connects the first sensor assembly and the first conductive line, and connects the second sensor assembly and the first conductive line.

The electronic device may include a first insulation layer disposed below the first sensor assembly and the second sensor assembly, and a second conductive line disposed on the first insulation layer and connecting the first sensor assembly and the signal processor, and connecting the second sensor assembly and the signal processor.

### BRIEF DESCRIPTION OF THE DRAWINGS

FIG. 1 is a schematic perspective view of an electronic device according to an embodiment.

FIG. 2 is a schematic cross-sectional view of the electronic device taken along a line A-A′ in FIG. 1.

FIG. 3 is a schematic top plan view of an electronic part included in an electronic device according to an embodiment.

FIG. 4 is a schematic cross-sectional view of a first sensor assembly taken along a line I-I′ in FIG. 3.

FIG. 5 is a schematic cross-sectional view of a second sensor assembly taken along a line II-II′ in FIG. 3.

FIG. 6 is a schematic top plan view of an electronic part included in an electronic device according to an embodiment.

FIG. 7 is a schematic cross-sectional view of a first sensor assembly taken along a line III-III′ in FIG. 6.

FIG. 8 is a schematic cross-sectional view of a second sensor assembly taken along a line IV-IV′ in FIG. 6.

FIG. 9 and FIG. 10 are schematic top plan views of an electronic part included in an electronic device according to an embodiment.

FIG. 11 is a schematic cross-sectional view of an electronic device according to an embodiment.

FIG. 12 is a schematic top plan view illustrating a signal processing process of an electronic part included in an electronic device according to an embodiment.

FIG. 13 is a schematic diagram illustrating a signal processing process of an electronic part included in an electronic device according to an embodiment.

FIGS. 14, 15, and 16 are graphs illustrating signal processing results of an electronic part included in an electronic device according to an embodiment.

FIG. 17 is a schematic diagram for illustrating a signal processing process of an electronic part included in an electronic device according to an embodiment.

FIG. 18 is a schematic block diagram of an electronic device according to an embodiment.

### DETAILED DESCRIPTION

The present disclosure will be described more fully hereinafter with reference to the accompanying drawings, in which exemplary embodiments of the disclosure are shown. As those skilled in the art would realize, the described embodiments may be modified in various different ways, without departing from the spirit or scope of the present disclosure.

Descriptions of parts not related to the present disclosure are omitted, and like reference numerals designate like elements throughout the specification.

Further, since sizes and thicknesses of constituent members shown in the accompanying drawings are arbitrarily given for better understanding and ease of description, the present disclosure is not limited to the illustrated sizes and thicknesses. In the drawings, the thickness of layers, films, panels, regions, etc., are exaggerated for clarity. In the drawings, for better understanding and ease of description, the thicknesses of some layers and areas are exaggerated.

It should be understood that when an element such as a layer, film, region, or substrate is referred to as being “on” another element, the element can be directly on the other element or intervening elements may also be present. In contrast, when an element is referred to as being “directly on” another element, there are no intervening elements present. Further, in the specification, the word “on” or “above” represents positioned on or below the object portion, and does not necessarily represent positioned on the upper side of the object portion based on a gravitational direction.

In addition, unless explicitly stated to the contrary, the word “comprise,” and variations such as “comprises” and “comprising,” should be understood to imply the inclusion of stated elements but not the exclusion of any other elements.

Further, throughout the specification, the phrase “on a plane” represents viewing a target portion from the top, and the phrase “in a cross-section” represents viewing a cross-section formed by vertically cutting a target portion from the side.

It will be understood that, although the terms “first,” “second,” etc. may be used herein to describe various elements, these elements should not be limited by these terms. These terms are used to distinguish one element from another element. For example, a first element discussed below could be termed a second element without departing from the teachings and spirit of the present disclosure. Similarly, the second element could also be termed the first element.

Embodiments of the present disclosure provide an electronic device including a first sensor assembly comprising a light emitter and a first optical sensor adjacent to the light emitter, and a second sensor assembly comprising a second optical sensor spaced apart from the light emitter and the first optical sensor. The first sensor assembly is configured to output a first detection signal corresponding to a combination of emitted light and ambient light reflected from a body part of a user. The second sensor assembly outputs a second detection signal corresponding to ambient light. A signal processor receives the first and second detection signals and generates a refined detection signal by reducing or removing the ambient light noise based on the differential information from the second detection signal. By detecting different lights using two different sensors, the electronic device enhances the detection signal even under dynamic conditions such as user motion or varying light environments.

In some aspects, the second optical sensor is spaced farther from the light emitter than the first optical sensor. This configuration minimizes influence from the light emitted from the light emitter of the first sensor assembly. The configuration allows the second sensor assembly to serve as a reference for ambient light variation, enabling real-time noise differentiation in the signal processor. Accordingly, the configuration of first and second sensor assembly enhances detection accuracy and reduces motion artifacts.

According to embodiments, errors or distortions that may be caused by an external light source in a signal measurement using light may be reduced. Accordingly, accuracy of the signal measurement using light may be enhanced.

In addition, according to embodiments, when measuring biological signals such as blood flow using light, noise due to an external light that may be generated by a body motion may be corrected. Accordingly, the accuracy of measuring biological signals such as blood flow measurement may be enhanced.

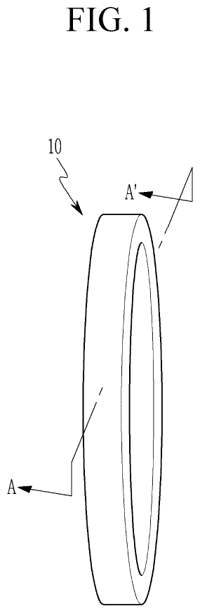

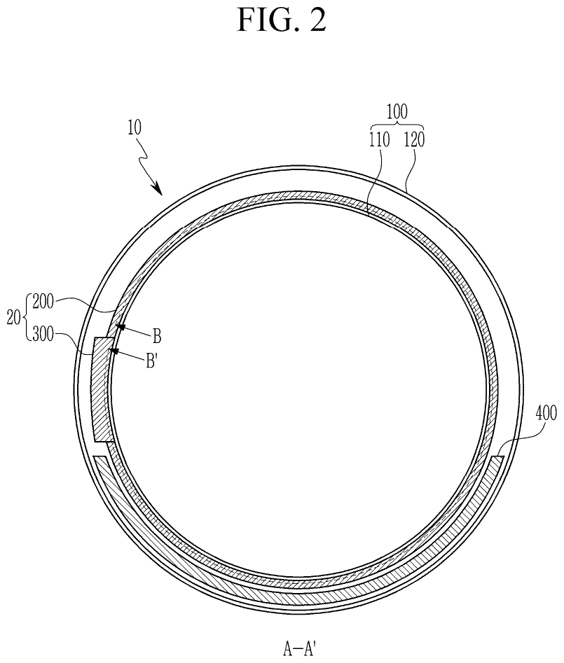

FIG. 1 is a schematic cross-sectional view of an electronic device according to an embodiment. FIG. 2 is a schematic cross-sectional view of an electronic device according to an embodiment. FIG. 2 is a cross-sectional view of the electronic device taken along a line A-A′ in FIG. 1.

Referring to FIG. 1 and FIG. 2, an electronic device 10 may be a wearable electronic device. For example, the electronic device 10 may have a shape that may be worn on a body part of a user.

The electronic device 10 may include a housing 100, an electronic part 20, and a battery 400. For example, the housing 100 may include an inner cover 110 and an outer cover 120. The housing 100 may have a ring shape in a cross-sectional view. The outer cover 120 may form the outer surface of the housing 100, and the inner cover 110 may form the inner surface of the housing 100. A finger of the user may be inserted into the inner surface of the housing 100, and at least a portion of the inner surface may come into contact with the finger. In some cases, the wrist of the user may be inserted into the inner surface of the housing 100, and at least a portion of the inner surface may come into contact with the wrist.

An electronic part 20 may be disposed in the housing 100. For example, the electronic part 20 may be positioned in the internal space formed by the outer cover 120 and the inner cover 110 of the housing 100. The electronic part 20 may include an optical sensor component 200 and a signal processor 300. The electronic device 10 may further include a battery 400.

The electronic device 10 may include the optical sensor component 200, the signal processor 300, and the battery 400 built into the housing 100. The optical sensor component 200 and the signal processor 300 may be physically connected. For example, one end of the optical sensor component 200 may be connected to one end of the signal processor 300. For example, a first end and a second end of the optical sensor component 200 may be connected to a first end and the a second of the signal processor 300, respectively. For example, the optical sensor component 200 may be connected to both ends of the signal processor 300, so that the optical sensor component 200 and the signal processor 300 have a ring shape. The optical sensor component 200 and the signal processor 300 may be connected to each other to form the single, inseparable ring-shaped electronic part. The optical sensor component 200 and the signal processor 300 may form the ring shape and may be positioned adjacent to the inner surface of the ring-shaped housing 100.

The battery 400 may be disposed between the optical sensor component 200 and the outer cover 120 of the housing 100. For example, the battery 400 may be spaced apart from the optical sensor component 200. The battery 400 may overlap at least a portion of the optical sensor component 200. For example, the battery 400 may overlap less than approximately 60%, 50%, or 40% of the total surface area of optical sensor component 200.

In some cases, the battery 400 might not overlap the signal processor 300. The signal processor 300 may be configured to have, for example, a driving chip, etc. Therefore, the signal processor 300 may have a greater thickness measured in a radial direction (e.g., measured from the outer surface direction of the outer cover 120 to the inner surface of the inner cover 110 of the housing 100) than the optical sensor component 200. The battery 400 is disposed to overlap the optical sensor component 200 without overlapping the signal processor 300, so that space efficiency inside the housing 100 may be enhanced, and the total thickness from the inner surface of the housing 100 to the outer surface direction (e.g., the radial direction) of the electronic device 10 may be reduced.

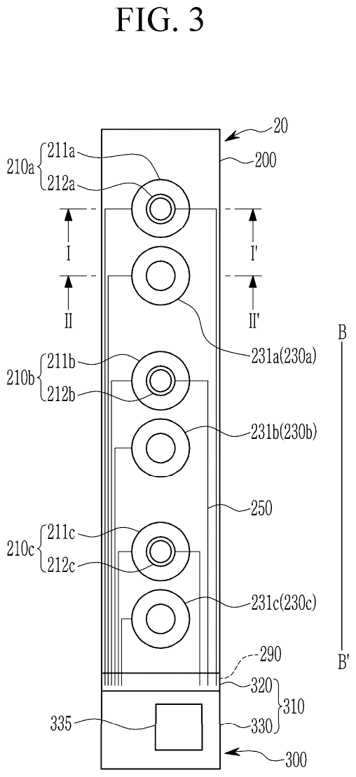

FIG. 3 is a schematic cross-sectional view of an electronic device according to an embodiment. FIG. 3 is a plan view of the electronic part 20 in a B-B′ region of FIG. 2, showing the optical sensor component 200 and signal processor 300.

Referring to FIG. 3, the electronic device 10 may include the optical sensor component 200 and the signal processor 300. The optical sensor component 200 and the signal processor 300 may be connected to each other. For example, one end of the optical sensor component 200 is connected to an end of the signal processor 300.

The signal processor 300 may include a printed circuit board (PCB) 310 and a sensor processor 330 disposed on the PCB 310. The PCB 310 may be flexible. The sensor processor 330 may be mounted on the PCB 310 in the form of an integrated circuit chip. The PCB 310 may include a pad part 320, and the pad part 320 may be connected to a pad part 290 of the optical sensor component 200. Accordingly, the optical sensor component 200 and the signal processor 300 may be electrically connected.

The sensor processor 330 may be disposed on a flexible printed circuit board (FPCB), and may include a driving chip 335. The driving chip 335 included in the sensor processor 330 may include various driving circuits for driving the electronic device 10 and connectors for a power supply. The signal processor 300 may apply driving signals to the optical sensor component 200 and process detection signals received from the optical sensor component 200.

The optical sensor component 200 may include first sensor assembly 210a, 210b, and 210c and second sensor assembly 230a, 230b, and 230c. The first sensor assembly 210a, 210b, and 210c may include first optical sensors 211a, 211b, and 211c and light emitters 212a, 212b, and 212c. The second sensor assembly 230a, 230b, and 230c may include second optical sensors 231a, 231b, and 231c. The second sensor assembly 230a, 230b, and 230c might not include the light emitters 212a, 212b, and 212c.

The first optical sensors 211a, 211b, and 211c may be adjacent to the light emitters 212a, 212b, and 212c. In some embodiments, the first optical sensors 211a, 211b, and 211c may surround the light emitters 212a, 212b, and 212c. The first optical sensors 211a, 211b, and 211c may detect light incident upon the object by reflection of external light and light incident upon the object by reflection of light emitted from the light emitters 212a, 212b, and 212c.

For example, the light may be emitted from the light emitters 212a, 212b, and 212c, and the light may be reflected from the object to the light emitters 212a, 212b, and 212c. The reflected light may be incident on the first optical sensors 211a, 211b, and 211c. The first optical sensors 211a, 211b, and 211c are disposed adjacent to the light emitters 212a, 212b, and 212c to detect two types of reflected light. First, the first optical sensors 211a, 211b, and 211c are configured to detect the light emitted from the light emitters 212a, 212b, and 212c and reflected from and incident on the object. Second, the first optical sensors 211a, 211b, and 211c are configured to detect external light reflected from and incident on the object. The first optical sensors 211a, 211b, and 211c may detect two types of the reflected light and output a first detection signal to the signal processor 300.

The second optical sensors 231a, 231b, and 231c may be further spaced apart from the light emitters 212a, 212b, and 212c than the first optical sensors 211a, 211b, and 211c. For example, the distance between the second optical sensor 231a and the light emitter 212a is greater than the distance between the second optical sensor 231a and the first optical sensor 211a. The second optical sensors 231a, 231b, and 231c may be configured to detect the external light incident and reflected from the object. For example, since the second optical sensors 231a, 231b, and 231c are positioned away from the light emitters 212a, 212b, and 212c, the amount of light emitted from the light emitters 212a, 212b, and 212c is reflected by the object and detected by the second optical sensors 231a, 231b, and 231c may be reduced or minimal.

The second optical sensors 231a, 231b, and 231c may detect light incident on the object due to external light being reflected by the object and output a second detection signal to the signal processor 300.

In an embodiment, the first optical sensors 211a, 211b, and 211c may surround the light emitters 212a, 212b, and 212c, respectively. The light emitters 212a, 212b, and 212c may be formed in a circular shape, for example, and the first optical sensors 211a, 211b, and 211c may be formed in a ring shape surrounding the circular light emitters 212a, 212b, and 212c. Accordingly, the first optical sensors 211a, 211b, and 211c may accurately detect light emitted from the light emitters 212a, 212b, and 212c and measure the detection signals.

Additionally, the second optical sensors 231a, 231b, and 231c may be formed in a ring shape and spaced apart from the first sensor assembly 210a, 210b, and 210c. Accordingly, the light detected by the second optical sensors 231a, 231b, and 231c from the light emitted from the light emitters 212a, 212b, and 212c may be reduced. In some cases, the first optical sensors 211a, 211b, and 211c and the second optical sensors 231a, 231b, and 231c may have substantially the same shape and dimension. However, embodiments are not necessarily limited thereto. For example, the first optical sensors 211a, 211b, and 211c and the second optical sensors 231a, 231b, and 231c may have different shapes or dimensions.

The first optical sensors 211a, 211b, and 211c and the second optical sensors 231a, 231b, and 231c may have substantially the same shape. Accordingly, first optical sensors 211a, 211b, and 211c and the second optical sensors 231a, 231b, and 231c may be manufactured using a same mask. For example, a type of a mask (e.g., a fine metal mask (FMM)) for manufacture the optical sensor component 200 may be reduced, so that the manufacturing process may be simplified and the production cost may be reduced.

By adjusting the spacing between the light emitters 212a, 212b, and 212c, the first optical sensors 211a, 211b, and 211c and the second optical sensors 231a, 231b, and 231c differently, the first detection signal and the second detection signal may be outputted from the first sensor assembly 210a, 210b, and 210c and the second sensor assembly 230a, 230b, and 230c, respectively.

The first sensor assembly 210a, 210b, and 210c may measure the light from the outside (e.g., natural light or other light sources not from the light emitters 212a, 212b, and 212c) and the light emitted from the light emitters 212a, 212b, and 212c to output the first detection signal. The second sensor assembly 230a, 230b, and 230c may measure the external light (e.g., natural light or other light sources not from the light emitters 212a, 212b, and 212c) to output the second detection signal. Accordingly, noise caused by the light sourced from the outside may be measured more accurately through the first detection signal and the second detection signal.

The first optical sensors 211a, 211b, and 211c of the first sensor assembly 210a, 210b, and 210c may output the first detection signal. The first detection signal output from the first optical sensors 211a, 211b, and 211c may be transmitted to the signal processor 300 through a first conductive line 250. The second optical sensors 231a, 231b, and 231c of the second sensor assembly 230a, 230b, and 230c may output the second detection signal. The first detection signal output from the second optical sensors 231a, 231b, and 231c may be transmitted to the signal processor 300 through the first conductive line 250.

The signal processor 300 may receive the first detection signal and the second detection signal and generate a refined detection signal. The first detection signal and second detection signal may be differentially detected in the signal processor 300 to measure external light noise. If the electronic device is a wearable electronic device in the shape of a ring, a difference in the amount of the incident external light may occur due to muscle movement inside the finger when the finger moves. Therefore, external light noise may occur due to differences in the incident amount of the external light. For example, motion artifacts may be caused by the motion of the user.

However, according to an embodiment, the optical sensor component 200 may output the first detection signal and the second detection signal, and the first detection signal and the second detection signal may be differentially detected in the signal processor 300, thereby measuring the noise. For example, differential detection may involve subtracting the second detection signal from the first detection signal, where the second detection signal corresponds light detected by ambient or external light and the first detection signal corresponds to the light emitted from the light emitters. Accordingly, the external light noise may be measured more accurately, and the refined detection signal may be measured by correcting the signal measured through the measured external light noise. Accordingly, the motion artifacts may be reduced, and biological signals such as a pulse and a heart rate may be measured more accurately even when the user is in motion.

In some embodiments, the electronic part 20 may include a plurality of first sensor assembly 210a, 210b, and 210c and second sensor assembly 230a, 230b, and 230c. For example, as shown in FIG. 3, the electronic part 20 includes three first sensor assembly 210a, 210b, and 210c and three second sensor assembly 230a, 230b, and 230c. Accordingly, the external light noise may be accurately computed based on the detection signals to generate the refined detection signal with reduced motion artifacts. However, embodiments are not necessarily limited thereto. For example, the electronic part 20 may include one or more first sensor assembly and second sensor assembly.

The plurality of first sensor assembly 210a, 210b, and 210c and the plurality of second sensor assembly 230a, 230b, and 230c may be alternately disposed with respect to each other. For example, one second sensor assembly 230a, 230b, and 230c may be disposed between two first sensor assembly 210a, 210b, and 210c, and one first sensor assembly 210a, 210b, and 210c may be disposed between two second sensor assembly 230a, 230b, and 230c. One first sensor assembly 210a, 210b, and 210c and one second sensor assembly 230a, 230b, and 230c that are alternately adjacent to each other may form a pair of sensor units.

When there is a plurality of first sensor assembly 210a, 210b, and 210c and second sensor assembly 230a, 230b, and 230c, a plurality of paired sensor units may be formed. In the plurality of paired sensor units, by each independently measuring the first detection signal and the second detection signal, the noise may be removed and the refined detection signal may be generated. The refined detection signals measured from each of the plurality of paired sensor units may be compared and corrected in a signal processor. Accordingly, the signal processor may generate an accurate refined detection signal.

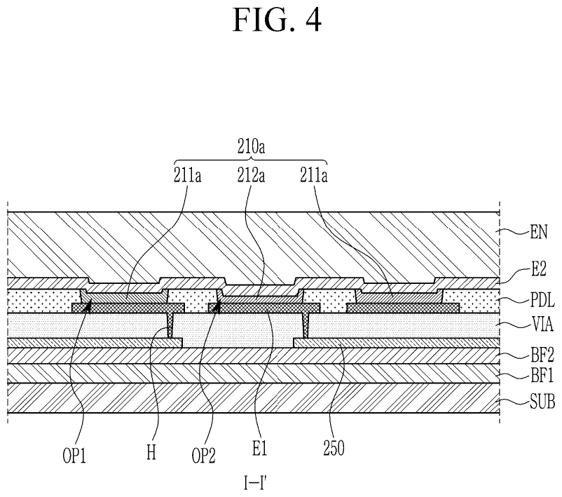

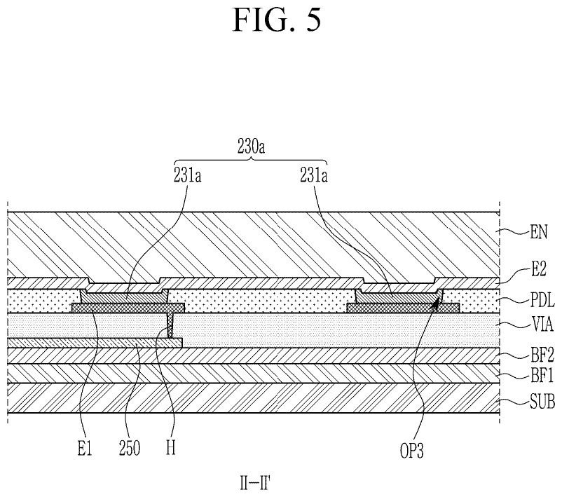

FIG. 4 is a schematic cross-sectional view of an electronic part included in an electronic device according to an embodiment. FIG. 4 is a cross-sectional view of a first sensor assembly taken along a line I-I′ of FIG. 3. FIG. 5 is a schematic cross-sectional view of an electronic part included in an electronic device according to an embodiment. FIG. 5 is a cross-sectional view of a second sensor assembly taken along a line II-II′ of FIG. 3.

Referring to FIG. 4 and FIG. 5, a first sensor assembly 210a may include a substrate SUB, a first optical sensor 211a, and a light emitter 212a. The first optical sensor 211a and the light emitter 212a may be disposed on the substrate SUB. The second sensor assembly 230a may include the substrate SUB and the second optical sensor 231a disposed on the substrate SUB.

In an embodiment, the substrate SUB may be disposed on the inner surface of the outer cover 120 of the housing 100 as described with reference to FIG. 1 and FIG. 2. The substrate SUB may be disposed adjacent to the outer surface of the housing 100. The first optical sensor 211a, the light emitter 212a, and the second optical sensor 231a disposed on the substrate SUB may be adjacent to the inner surface of the inner cover 110 of the housing 100.

The substrate SUB may be a flexible substrate. For example, the substrate SUB may be a film including a polymer resin such as polyimide, polyamide, or polyethylene terephthalate.

A first buffer layer BF1 may be disposed on the substrate SUB. A second buffer layer BF2 may be disposed on the first buffer layer BF1. The first buffer layer BF1 and the second buffer layer BF2 block impurities from the substrate SUB when forming the first sensor assembly 210a and the second sensor assembly 230a. Forming the first buffer layer BF1 and the second buffer layer BF2 enhances the characteristics of the first sensor assembly 210a and the second sensor assembly 230a, and planarizes the surface of the substrate SUB to reduce the stress applied when the optical sensor component is bent. Since the buffer layer is formed of a multi-layer structure including the first buffer layer BF1 and the second buffer layer BF2, the stress applied to the optical sensor component may be further reduced.

The first buffer layer BF1 and the second buffer layer BF2 may include an inorganic insulating material such as silicon nitride (SiNₓ), silicon oxide (SiOₓ), silicon oxynitride (SiOₓN_{y}).

The first buffer layer BF1 and the second buffer layer BF2 may include amorphous silicon. FIG. 4 and FIG. 5 illustrate that the buffer layers BF1 and BF2 are formed as a two-layer structure, but the buffer layers may be a single-layer structure or a multi-layer structure including three or more layers.

The first conductive line 250 may be disposed on the substrate SUB or the buffer layers BF1 and BF2. In an embodiment, the first conductive line 250 may be disposed on the second buffer layer BF2. The first conductive line 250 may connect the first sensor assembly 210a and the signal processor 300. The first conductive line 250 may connect the second sensor assembly 230a and the signal processor 300. The first conductive line 250 may function as a signal line, a data line, a scan line, or similar interconnect. For example, the first conductive line 250 may include aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), copper (Cu), or other metallic elements or alloys.

A first insulation layer VIA may be disposed on the substrate SUB or the buffer layers BF1 and BF2. The first insulation layer VIA may be a planarization layer. For example, the first insulation layer VIA may be disposed on and cover the second buffer layer BF2 and the first conductive line 250. For example, the first insulation layer VIA may include organic insulating materials such as common polymers such as poly-(methyl methacrylate) and polystyrene, polymer derivatives having phenolic groups, acryl-based polymers, imide-based polymers (e.g., polyimide), and siloxane-based polymers. The first insulation layer VIA may include a connection hole H that overlaps the first conductive line 250. For example, the connection hole H may penetrate the first insulation layer VIA to expose a portion of the first conductive line 250.

A first electrode E1 may be disposed on the first insulation layer VIA. The first electrode E1 may overlap the connection hole H of the first insulation layer VIA. A portion of the first electrode E1 may be disposed to overlap at least a portion of the connection hole H on the first insulation layer VIA. The first electrode E1 may function as an anode. The first electrode E1 may include metals such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), and copper (Cu). The first electrode E1 may include a transparent conductive oxide (TCO) such as indium tin oxide (ITO) or indium zinc oxide (IZO). The first electrode E1 may be a single-layer structure including a metallic material or a transparent conductive oxide, or a multi-layer structure including a metallic layer and a transparent conductive oxide.

The first electrode E1 may connect the first optical sensor 211a and the first conductive line 250. For example, the first optical sensor 211a may be disposed on the first electrode E1. The first electrode E1 may connect the second optical sensor 231a and the first conductive line 250. For example, the second optical sensor 231a may be disposed on the first electrode E1. The first electrode E1 may connect the light emitter 212a of the first sensor assembly 210a and the first conductive line 250. For example, the light emitter 212a may be disposed on the first electrode E1.

According to an embodiment, the first optical sensor 211a of the first sensor assembly 210a may be formed to surround the light emitter 212a. Therefore, if the first conductive line 250 is in direct contact with the first optical sensor 211a and the light emitter 212a, circuit damage or a fire may occur due to a short circuit between the first conductive line 250 connected to the first optical sensor 211a and the first conductive line 250 connected to the light emitter 212a. However, by connecting the first optical sensor 211a and the first conductive line 250 via the first electrode E1, and the light emitter 212a and the first conductive line 250 via the first electrode E1, the first conductive lines 250 may be electrically isolated to prevent short-circuiting. Therefore, the durability of the optical sensor component may be enhanced.

A second insulation layer PDL may be disposed on the first insulation layer VIA. The second insulation layer PDL may spatially separate and encapsulate the first optical sensor 211a, the light emitter 212a, and the second optical sensor 231a. The second insulation layer PDL may include a first opening OP1 that overlaps the first electrode E1 of the first optical sensor 211a, a second opening OP2 that overlaps the first electrode E1 of the light emitter 212a, and a third opening OP3 that overlaps the first electrode E1 of the second optical sensor 231a. The second insulation layer PDL may cover the edge of the first electrode E1. In some cases, the second insulation layer PDL may cover an upper surface of the first insulation layer VIA and at least a portion of an upper surface and the side surfaces of the first electrode E1.

The second insulation layer PDL may include an organic insulating material. The second insulation layer PDL may include an inorganic insulating material such as silicon oxynitride (SiOₓN_{y}) or silicon oxide (SiOₓ). The second insulation layer PDL may include both organic insulating material and inorganic insulating material.

The second insulation layer PDL may include a light blocking material and the color of the second insulation layer PDL may be formed in black. The light blocking material may include a resin or a paste including a carbon black, a carbon nanotube, a black dye, metal particles such as nickel, aluminum, molybdenum, and an alloy thereof, metal oxide particles such as chromium oxide, and metal nitride particles such as chromium nitride. If the second insulation layer PDL includes the light blocking material, reflection of the external light from the metal structures placed under the second insulation layer PDL may be reduced.

The light emitter 212a, which overlaps the second opening OP2 of the second insulation layer PDL, may include light emitting layers for emitting light. For example, the light emitter 212a may emit light. The light emitter 212a may include an organic light emitting diode. The light emitter 212a may include an inorganic light emitting diode.

The light emitter 212a may be adjacent to the inner surface of the inner cover 110 of the housing 100 as described with reference to FIG. 1. The light emitter 212a is disposed on the inner surface of the housing 100 and may emit light toward the inner side of the inner cover 110. The light emitter 212a may emit light toward the inside of the inner cover 110 of the housing 100, and thus may emit light toward, for example, a finger of a user of the electronic device 10. Light emitted from the light emitter 212a may be reflected from the body part of the user, and detected and received by the first optical sensor 211a. Accordingly, the first optical sensor 211a may receive the external light and the light emitted from the light emitter 212a and output the first detection signal. Even if the light emitted from the light emitter 212a is reflected from the body part of the user, the light might not enter the second optical sensor 231a based on the configuration of the first sensor assembly 210a and the second sensor assembly 230a. Accordingly, the second optical sensor 231a may receive the external light and output the second detection signal.

A function layer may be disposed on the light emitter 212a. The function layer may include an electron transport layer, a hole injection layer (HIL), a hole transport layer, etc.

The first optical sensor 211a, which overlaps the first opening OP1 of the second insulation layer PDL, may detect the light (e.g., ambient light or external light and reflected light emitted from the light emitter 212a) incident on the first optical sensor 211a and output the first detection signal. The second optical sensor 231a, which overlaps the third opening OP3 of the second insulation layer PDL, may detect the light incident on the second optical sensor 231a and output the second detection signal.

A second electrode E2 may be disposed on the second insulation layer PDL, the first optical sensor 211a, the light emitter 212a, and the second optical sensor 231a. The second electrode E2 may be cover upper surfaces of the second insulation layer PDL, the first optical sensor 211a, the light emitter 212a, and the second optical sensor 231a. For example, the second electrode E2 may be formed integrally without being separated. Accordingly, the second electrode E2 remains continuous, and enabling a uniform full-surface conduction.

The second electrode E2 may include a transparent conductive oxide, such as indium tin oxide (ITO) or indium zinc oxide (IZO). The second electrode E2 may include a reflective metal such as aluminum (Al), platinum (Pt), palladium (Pd), silver (Ag), magnesium (Mg), gold (Au), nickel (Ni), neodymium (Nd), iridium (Ir), chromium (Cr), calcium (Ca), molybdenum (Mo), titanium (Ti), tungsten (W), or copper (Cu). The second electrode E2 may be a single-layer structure including a transparent conductive oxide or a metallic material or a multi-layer structure including a transparent conductive oxide layer and a metallic layer.

A capping layer may be disposed on the second electrode E2. The capping layer may enhance the efficiency of the light emitted from the light emitter 212a by adjusting a refractive index. The capping layer may cover the second electrode E2. The capping layer may include an organic insulating material or may include an inorganic insulating material.

An encapsulation layer EN may be disposed on the capping layer or the second electrode E2. The encapsulation layer EN may encapsulate the first optical sensor 211a, the light emitter 212a, the second optical sensor 231a, and other components, to prevent moisture or oxygen from penetrating from the outside. The encapsulation layer EN may be formed of one or more layers. For example, the encapsulation layer EN may be a thin-film encapsulation layer including one or more inorganic layers and one or more organic layers. In some cases,

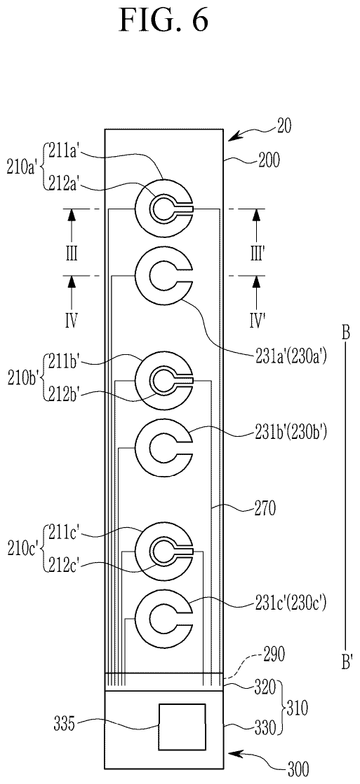

FIG. 6 is a schematic cross-sectional view of an electronic device according to an embodiment. FIG. 6 is a plan view of the region B-B′ of FIG. 2, showing the optical sensor component 200 and the signal processor 300, excluding the battery 400 among the electronic part 20 positioned inside the housing 100. In explaining FIG. 6, the explanation of configurations identical or similar to the configuration explained with reference to FIG. 3 may be omitted.

Referring to FIG. 6, detection signals may be transmitted to the signal processor 300 via a second conductive line 270 extending from the optical sensor component 200 to the signal processor 300.

The first detection signal output from the first optical sensors 211a′, 211b′, and 211c′ of the first sensor assembly 210a′, 210b′, and 210c′ may be transmitted to the signal processor 300 through the second conductive line 270. The second detection signal output from the second optical sensors 231a′, 231b′, and 231c′ of the second sensor assembly 230a′, 230b′, and 230c′ may be transmitted to the signal processor 300 through the second conductive line 270.

The optical sensor component 200 may include the first sensor assembly 210a′, 210b′, and 210c′ and the second sensor assembly 230a′, 230b′, and 230c′. The first sensor assembly 210a′, 210b′, and 210c′ may include the first optical sensors 211a′, 211b′, and 211c′ and the light emitters 212a′, 212b′, and 212c′, respectively. The second sensor assembly 230a′, 230b′, and 230c′ may include the second optical sensors 231a′, 231b′, and 231c′, respectively. The second sensor assembly 230a′, 230b′, and 230c′ might not include the light emitters 212a′, 212b′, and 212c′, respectively.

The first optical sensors 211a′, 211b′, and 211c′ may partially surround the light emitters 212a′, 212b′, and 212c′, respectively. The light emitters 212a′, 212b′, and 212c′ may, for example, have a shape of a light bulb. The light emitters 212a′, 212b′, and 212c′ may have a combination of a rectangular shape and a circular shape and may be formed into a circular shape with a protruded side surface. The first optical sensors 211a′, 211b′, and 211c′ may surround the circular shaped portion of the light emitters 212a′, 212b′, and 212c′. For example, the first optical sensors 211a′, 211b′, and 211c′ may surround a glass tube portion of the bulb shape of the light emitters 212a′, 212b′, and 212c′. For example, the first optical sensors 211a′, 211b′, and 211c′ may be “C”-shaped. Accordingly, process efficiency may be further enhanced.

The second optical sensors 231a′, 231b′, and 231c′ may have substantially the same shape as the first optical sensors 211a′, 211b′, and 211c′. For example, the second optical sensors 231a′, 231b′, and 231c′ may be formed in a ring shape with the sides opened (or having a shape of “C”), and spaced apart from the first sensor assembly 210a′, 210b′, and 210c′, respectively. Accordingly, the number of masks for manufacturing the optical sensor component 200 may be reduced, thereby enhancing process efficiency. The second optical sensors 231a′, 231b′, and 231c′ may have substantially the same planar shape as the first optical sensors 211a′, 211b′, and 211c′. In some embodiments, the second optical sensors 231a′, 231b′, and 231c′ may have a different planar shape than the first optical sensors 211a′, 211b′, and 211c′. For example, the second optical sensors 231a′, 231b′, and 231c′ may have a donut shape or ring shape.

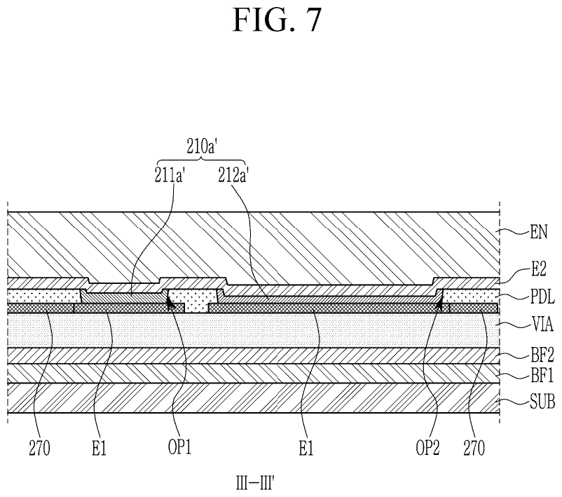

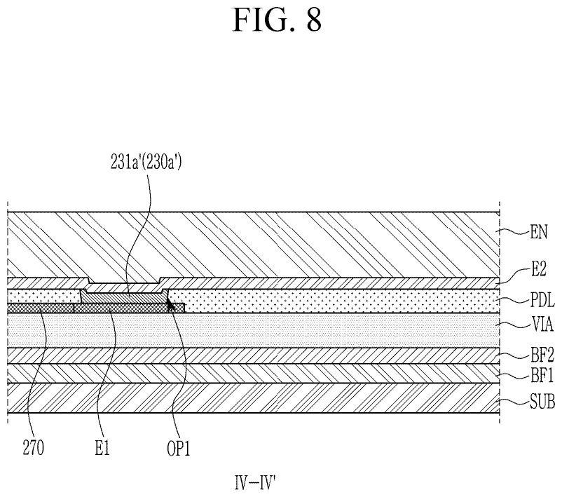

FIG. 7 is a schematic cross-sectional view of an electronic part included in an electronic device according to an embodiment. FIG. 7 is a cross-sectional view of a first sensor assembly taken along a line III-III′ of FIG. 6. FIG. 8 is a schematic cross-sectional view of an electronic part included in an electronic device according to an embodiment. FIG. 8 is a cross-sectional view of a second sensor assembly taken along a line IV-IV′ of FIG. 6.

Referring to FIG. 7 and FIG. 8, buffer layers BF1 and BF2 may be disposed on a substrate SUB, and a first insulation layer VIA may be disposed on the buffer layers BF1 and BF2. The first insulation layer VIA might not include a connecting hole compared to the configuration shown in FIGS. 4 and 5. A second insulation layer PDL may be disposed on the first insulation layer VIA. The second insulation layer PDL may include the openings OP1, OP2, and OP3. The openings OP1, OP2, and OP3 of the second insulation layer PDL may overlap the first electrode E1 disposed on the first insulation layer VIA. The first opening OP1 may overlap the first optical sensor 211a′. The second opening OP2 may overlap the light emitter 212a′. The third opening OP3 may overlap the second optical sensor 231a′. In regions where the first optical sensor 211a′, the light emitter 212a′, and the second optical sensor 231a′ are not disposed, the openings OP1, OP2, and OP3 might not be formed.

The first electrode E1 may be disposed on the first insulation layer VIA. The first optical sensor 211a′, the light emitter 212a′, and the second optical sensor 231a′ may be directly connected to the corresponding first electrode E1 in the region overlapping the openings OP1, OP2, and OP3 of the second insulation layer PDL. The first electrode E1 may be connected to the second conductive line 270. The first electrode E1 is directly connected to the first optical sensor 211a′, the light emitter 212a′, and the second optical sensor 231a′, and the first electrode E1 is directly connected to the second conductive line 270, so that the first optical sensor 211a′, the light emitter 212a′, and the second optical sensor 231a′ may each be connected to a signal processor.

In some cases, the second conductive line 270 may be formed in the same layer or level as the first electrode E1. For example, the second conductive line 270 and the first electrode E1 may be part of the same conductive layer, enabling direct electrical connectivity without vertical interconnections such as vias between different metal layers as shown In FIGS. 4-5. Accordingly, the manufacturing process can be enhanced.

According to an embodiment, the first optical sensor 211a′ of the first sensor assembly 210a′ may partially surround the light emitter 212a′. The light emitter 212a′ may have a bulb shape. The light emitter 212a′ may be formed as a combination of circular and rectangular shapes, such as a circular shape with a protruded side. The first optical sensor 211a′ may surround the circular-shaped portion of the light emitter 212a′. For example, the first optical sensor 211a′ may surround the glass tube portion of the bulb shape of the light emitter 212a′. For example, the first optical sensor 211a′ may be formed in a roughly “C” shaped structure. Therefore, even if the first electrode E1 is directly connected to the first optical sensor 211a′ and the light emitter 212a′, and the first electrode E1 is connected to the second conductive line 270, a short circuit between the second conductive lines 270 might not occur. In addition, when connecting the first sensor assembly 210a′ and the second sensor assembly 210b′ to the signal processor 300, the second conductive line 270 may be used without additional conductive lines. Accordingly the manufacturing process of the optical sensor component may be simplified.

In an embodiment, the first sensor assembly and the second sensor assembly may be used as a combination of the first optical sensor 211a, the light emitter 212a, and the second optical sensor 231a described with reference to FIG. 3, and the first optical sensor 211a′, the light emitter 212a′ and the second optical sensor 231a′ described with reference to FIG. 6.

The first sensor assembly of the optical sensor component may have the shape of the first sensor assembly 210a described with reference to FIG. 3, and the second sensor assembly of the optical sensor component may have the shape of the second sensor assembly 230a′ described with reference to FIG. 6.

The first sensor assembly 210a of the optical sensor component may include the substrate SUB and the first conductive line 250 disposed on the substrate SUB and connecting the first sensor assembly 210a to a signal processor. Additionally, the first sensor assembly 210a may include the first insulation layer VIA disposed on the first conductive line 250 and including the connection hole H and the first electrode E1 overlapping the connection hole H. The first electrode E1 may connect the first optical sensor 211a and the light emitter 212a to the first conductive line 250.

The second sensor assembly 230a′ of the optical sensor component may include the substrate SUB, the first insulation layer VIA disposed on the substrate SUB, the first electrode E1 disposed on the first insulation layer VIA, the second conductive line 270 connected to and adjacent to the first electrode E1, and the second insulation layer PDL. In one aspect, the second conductive line 270, the first electrode E1, and the second insulation layer PDL are disposed on the first insulation layer VIA. The first electrode E1 may overlap the third opening OP3 formed in the second insulation layer PDL. The second insulation layer PDL may cover an upper surface of the second conductive line 270 and at least a portion of an upper surface and the side surface of the first electrode E1. The other side surface (or the opposite side surface) of the first electrode E1 may be in contact with a side surface of the second conductive line 270.

The first electrode E1 and the second conductive line 270 may be disposed in the same layer or level. The first electrode E1 and the second conductive line 270 may be formed using the same mask (e.g., a fine metal mask) in the same process and may include substantially the same material.

In an embodiment, the first sensor assembly of the optical sensor component may have the shape of the first sensor assembly 210a′ described with reference to FIG. 6, and the second sensor assembly of the optical sensor component may have the shape of the second sensor assembly 230a described with reference to FIG. 3.

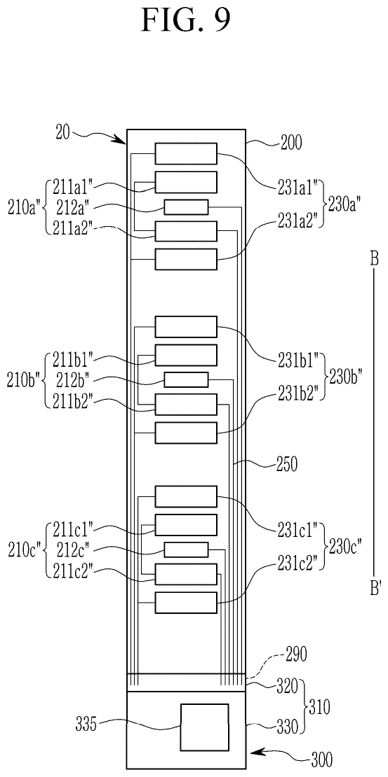

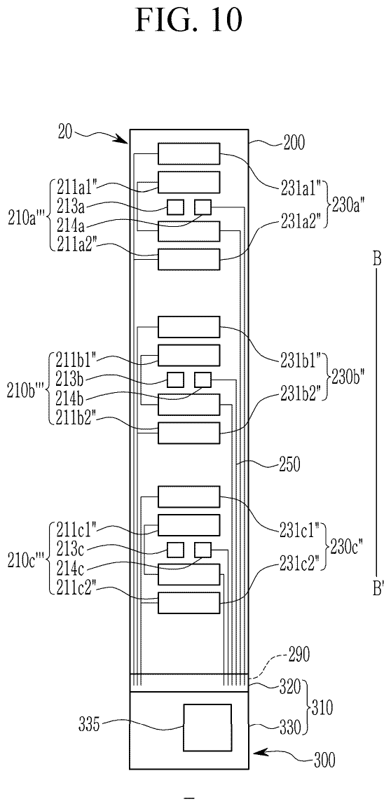

FIG. 9 and FIG. 10 are schematic top plan views of an electronic part included in an electronic device according to an embodiment. FIG. 9 and FIG. 10 are top plan views of the region B-B′ in FIG. 2. In describing FIG. 9 and FIG. 10, description of configurations identical or similar to those described with reference to FIG. 3 and FIG. 6 may be omitted.

Referring to FIG. 9, the first optical sensor may include the first parts 211a1″, 211b1″, and 211c1″ and the second parts 211a2″, 211b2″, and 211c2″ which are each disposed on both sides of the light emitters 212a″, 212b″, and 212c″, respectively, and separated from each other. For example, the first parts 211a1″, 211b1″, and 211c1″ may represent the configuration of the first optical sensor disposed on one side of the light emitters 212a″, 212b″, and 212c″, and the second parts 211a2″, 211b2″, and 211c2″ may represent the configuration of the first optical sensor disposed on the opposite side of the light emitters 212a″, 212b″, and 212c″. The first parts 211a1″, 211b1″, and 211c1″ and the second parts 211a2″, 211b2″, and 211c2″ may be spaced apart from each other with the light emitters 212a″, 212b″, and 212c″ interposed therebetween. The first parts 211a1″, 211b1″, and 211c1″ and the second parts 211a2″, 211b2″, and 211c2″ may be disposed adjacent to the light emitters 212a″, 212b″, and 212c″. The first parts 211a1″, 211b1″, and 211c1″ and the second parts 211a2″, 211b2″, and 211c2″ may form the first optical sensor.

The second optical sensor may be disposed and spaced apart from the light emitters 212a″, 212b″, and 212c″ with the first optical sensor interposed therebetween. The second optical sensor may include third parts 231a1″, 231b1″, and 231c1″ and fourth parts 231a2″, 231b2″, and 231c2″. For example, the third parts 231a1″, 231b1″, and 231c1″ may represent the configuration of the second optical sensor disposed on one side of the light emitters 212a″, 212b″, and 212c″, and the fourth parts 231a2″, 231b2″, and 231c2″ may represent the configuration of the second optical sensor disposed on the opposite side of the light emitters 212a″, 212b″, and 212c″. The third parts 231a1″, 231b1″, and 231c1″ may be disposed and spaced apart from the light emitters 212a″, 212b″, and 212c″ with the first parts 211a1″, 211b1″, and 211c1″ interposed therebetween. The fourth parts 231a2″, 231b2″, and 231c2″ may be disposed and spaced apart from the light emitters 212a″, 212b″, and 212c″ with the second parts 211a2″, 211b2″, and 211c2″ interposed therebetween. Accordingly, the configuration of the optical sensors may be simplified, thereby reducing the complexity of the manufacturing process of the optical sensor component.

In an embodiment, the first sensor assembly 210a″ of the optical sensor component 200 includes a light emitter 212a″ disposed between two components of the optical sensor, which are a first part 211a1″ and a second part 211a2″. The first part 211a1″ is disposed above the light emitter 212a″, and the second part 211a2″ is disposed below the light emitter 212a″ in the plan view (or top view), and both parts are spaced apart from the light emitter 212a″. Additionally, a second sensor assembly 230a″ is disposed outwardly relative to the first sensor assembly 210a″. The second sensor assembly 230a″ includes a third part 231a1″ and a fourth part 231a2″. The third part 231a1″ is disposed above the first part 211a1″, and the fourth part 231a2″ is disposed below the second part 211a2″ in the plan view (or top view). The second optical sensor is spaced from the light emitter 212a″ with the first optical sensor interposed therebetween. In the direction toward the signal processor 300, the components may be sequentially arranged as the third part 231a1″ of the second optical sensor, the first part 211a1″ of the first optical sensor, the light emitter 212a″ of the first optical sensor, the second part 211a2″ of the first optical sensor, and the fourth part 231a2″ of the second optical sensor.

The first optical sensor (including the first parts 211a1″, 211b1″, and 211c1″ and the second parts 211a2″, 211b2″, and 211c2″), light emitters 212a″, 212b″, and 212c″ and the second optical sensors 231a″, 231b″, and 231c″ may be connected to the pad part 320 of the signal processor 300 via the first conductive line 250. The first parts 211a1″, 211b1″, and 211c1″ and the second parts 211a2″, 211b2″, and 211c2″ of the first optical sensor may each be connected to the pad part 320 of the signal processor 300 via the first conductive line 250. The light emitters 212a″, 212b″, and 212c″ may be connected to the pad part 320 of the signal processor 300 via the first conductive line 250. The third parts 231a1″, 231b1″, and 231c1″ and the fourth parts 231a2″, 231b2″, and 231c2″ of the second optical sensor may each be connected to the pad part 320 of the signal processor 300 via the first conductive line 250. In an embodiment, the first parts 211a1″, 211b1″, and 211c1″ and the second parts 211a2″, 211b2″, and 211c2″ of the first optical sensor, the light emitters 212a″, 212b″, 212c″, and the third parts 231a1″, 231b1″, and 231c1″ and the fourth parts 231a2″, 231b2″, and 231c2″ of the second optical sensor may be connected to the first conductive line 250 via the first electrode E1, respectively.

Although the first optical sensor, the light emitters 212a″, 212b″, and 212c″ and the second optical sensor are connected to the signal processor 300 through the first conductive line 250, the first optical sensor, the light emitters 212a″, 212b″, 212c″, and the second optical sensor may be connected to the signal processor 300 via the first electrode E1 and/or the second conductive line 270 as described with reference to FIG. 7 and FIG. 8. For example, the first parts 211a1″, 211b1″, and 211c1″ and the second parts 211a2″, 211b2″, and 211c2″ of the first optical sensor, the light emitters 212a″, 212b″, and 212c″, and the third parts 231a1″, 231b1″, and 231c1″ and the fourth parts 231a2″, 231b2″, and 231c2″ of the second optical sensor may each be connected to the signal processor 300 via the second conductive line 270.

Referring to FIG. 10, there may be a plurality of light emitters, and the plurality of light emitters may emit light of at least two different wavelengths. For example, the plurality of light emitters may emit different lights. The light emitter may include first sub-light emitters 213a, 213b, and 213c that emit light of a first wavelength and second sub-light emitters 214a, 214b, and 214c that emit light of a second wavelength different from the first wavelength. The first light subb-emitters 213a, 213b, and 213c and the second sub-light emitters 214a, 214b, and 214c may be disposed between the first optical sensors of the first sensor assembly 210a″, 210b″, and 210c″. For example, the first sub-light emitters 213a, 213b, and 213c and the second sub-light emitters 214a, 214b, and 214c may be disposed between the first part and the second part of the first optical sensor. By using multiple wavelengths, the electronic device 10 can accurately detect errors resulting from the muscle motion of the user.

The optical sensor component and the signal processor described above may be disposed inside the housing 100 of the electronic device described with reference to FIG. 2 with an electronic part such as a battery 400. In FIG. 2, the optical sensor component 200 and the signal processor 300 are connected and disposed inside the housing in a ring shape, and the battery 400 overlaps a part of the optical sensor component 200, but the arrangement configuration of other electronic parts such as the battery 400 is not necessarily limited thereto.

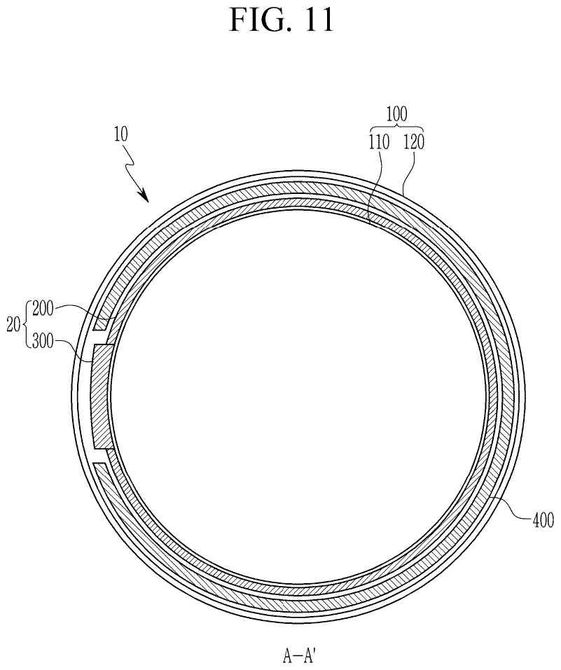

FIG. 11 is a schematic cross-sectional view of an electronic device according to an embodiment.

Referring to FIG. 11, the optical sensor component 200 and the signal processor 300 may be connected to each other in a ring shape configuration. The ring-shaped optical sensor component 200 and the signal processor 300 may be disposed adjacent to the inner surface of the inner cover 110 of the housing 100. For example, the inner surface of the inner cover 110 of the housing 100 may be referred to as the surface facing the internal components of the electronic device 10, and opposite to the surface in contact with the body part of the user. Additionally, the surface that contacts the body part of the user may be referred to as the outer surface of the inner cover 110 of the housing 100.

The battery 400 may be placed adjacent to the inner surface of the outer cover 120 of the housing 100 while overlapping the optical sensor component 200. The battery 400 may overlap a majority region of the optical sensor component 200. For example, the battery 400 may be disposed to overlap about 70%, 80%, or about 90% or more of the entire surface area of the optical sensor component 200. Accordingly, the capacity of the battery 400 may be increased, which may increase the usable time of the electronic device 10. In some aspects, the inner surface of the outer cover 120 may be opposite from the inner surface of the inner cover 110, and may face inward toward the internal electronic components, including the battery 400 and the optical sensor component 200.

The battery 400 may be positioned so as not to overlap the signal processor 300. Since a driving chip or other components may be mounted on the signal processor 300, the thickness of the housing 100 measured in the radial direction may be greater than the thickness of the optical sensor component 200. By positioning the battery 400 so that the battery does not overlap the signal processor 300, the thickness of the electronic device 10 measured in the radial direction may be reduced.

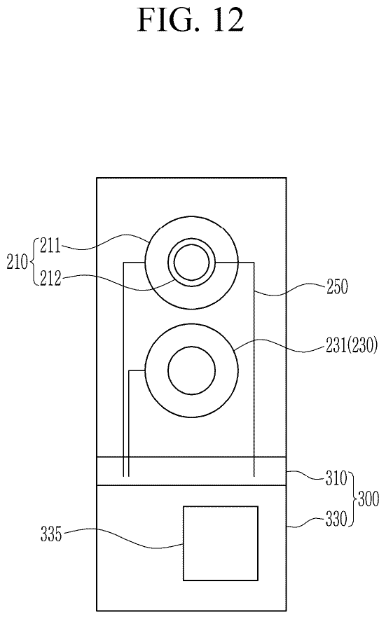

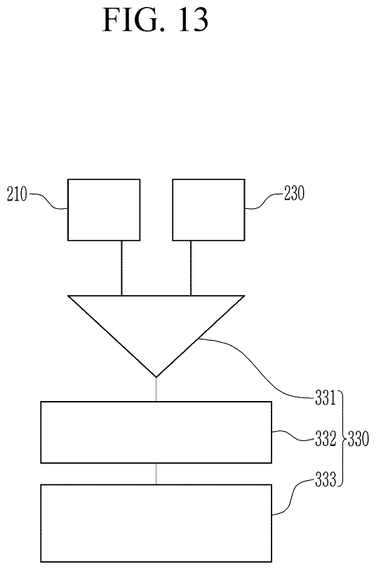

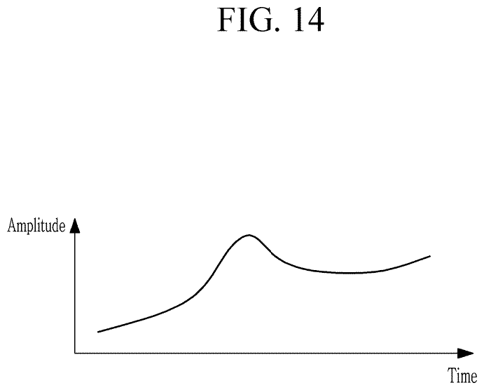

FIG. 12 is a schematic top plan view for illustrating a signal processing process of an electronic part included in an electronic device according to an embodiment. FIG. 13 is a schematic diagram for illustrating a signal processing process of an electronic part included in an electronic device according to an embodiment. FIGS. 14-16 are graphs showing signal processing results of an electronic part included in an electronic device according to an embodiment. FIG. 12-FIG. 16 are examples of an electronic device including an optical sensor component that includes a pair of sensor units (a combination of the first sensor assembly and the second sensor assembly).

Referring to FIG. 12 and FIG. 13, the first sensor assembly 210 may output the first detection signal. The first optical sensor 211 may detect light. The light emitter 212 of the first sensor assembly 210 may emit light. Light emitted from the light emitter 212 may be reflected from the body part of a user and incident on the first optical sensor 211. The first optical sensor 211 may detect the light emitted from the light emitter 212 and the light incident due to reflection of external light from the body part of the user. The first sensor assembly 210 may output a first detection signal through the light detected by the first optical sensor 211. The first detection signal output from the first sensor assembly 210 may be transmitted to the signal processor 300 through the first conductive line 250.

The second sensor assembly 230 may output a second detection signal. The second optical sensor 231 may detect the light. The second optical sensor 231 may detect incident light as external light is reflected from the body part of the user. The second sensor assembly 230 may output the second detection signal through the light detected by the second optical sensor 231. The second detection signal output from the second sensor assembly 230 may be transmitted to the signal processor 300 through the first conductive line 250.

The first detection signal and the second detection signal transmitted to the signal processor 300 may be combined in the signal assembly unit 331 of the sensor processor 330. In some embodiments, the first detection signal and the second detection signal may be individually processed in the signal assembly unit 331. The first detection signal and second detection signal combined in the signal assembly unit 331 may each be converted into visible data (e.g., data in a graph format) in the signal converter 332.

The noise of the first detection signal converted into data and the second detection signal converted into data may be adjusted in the signal analysis unit 333. The first detection signal may include a mixture of the signal generated by the light emitter 212 and the signal caused by the external light. The second detection signal may be a signal caused by the external light. Therefore, by excluding the signal caused by the external light included in both the first detection signal and the second detection signal, the noise may be removed. Accordingly, the signal processor 300 may generate a refined signal.

FIGS. 14-16 are graphs illustrating signal processing results of an electronic part included in an electronic device according to an embodiment. For example, FIGS. 14-16 show a signal amplitude (a PPG signal amplitude) based on photoplethysmography (PPG) as data by sampling a detection signal output during user movement while the user wears the electronic device.

Referring to FIG. 14, the first detection signal, which includes the PPG signal generated based on the light emitter and the PPG signal generated based on the external environment, might not accurately detect the peak. The peak represents basic information for interpreting the PPG signal. Therefore, it may be difficult to obtain accurate PPG biometric information using the first detection signal, as it is difficult to distinguish whether the PPG signal is a systolic peak, a diastolic peak, a peak caused by arrhythmia, or a peak caused by the external light noise.

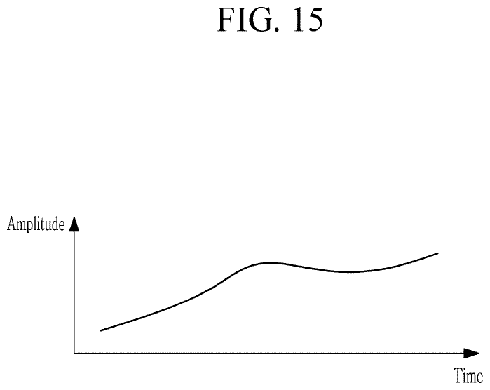

However, referring to FIG. 15, the second detection signal, which includes the signals generated based on the external light, samples external light information according to the user movement every hour. As a result, noise information based on the external light is sensed.

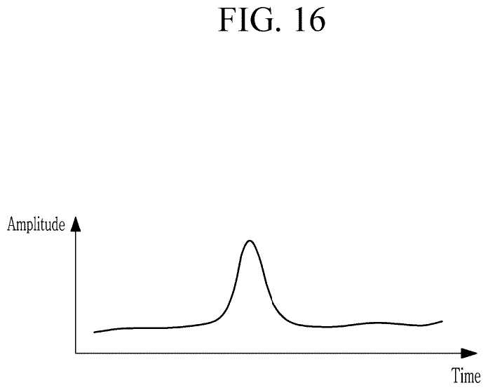

Referring to FIG. 16, the PPG signal is generated based on the light emitter with the external light noise removed. This refined signal may be obtained by differentially sensing the first detection signal and the second detection signal in real time. Using both the first detection signal and the second detection signal enables isolation of valid biometric data, resulting in improved signal clarity and measurement accuracy.

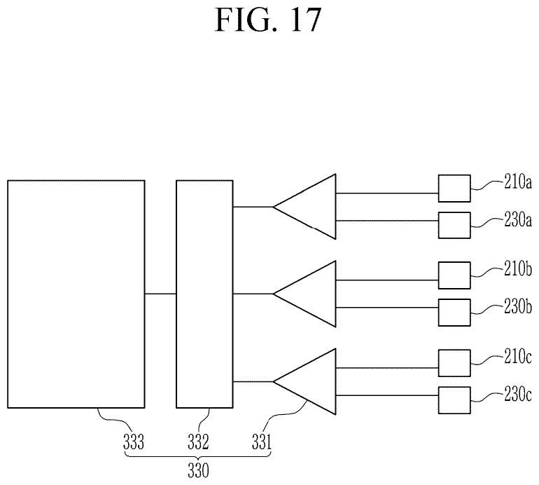

FIG. 17 is a schematic diagram for illustrating a signal processing process of an electronic part included in an electronic device according to an embodiment. FIG. 17 is an example of an electronic device including an optical sensor component that includes three pairs of paired sensor units (a combination of the first sensor assembly and the second sensor assembly). However, the number of paired sensor units are not necessarily limited thereto.

Referring to FIG. 17, the first sensor assembly 210a, 210b, and 210c may each output a first detection signal. The second sensor assembly 230a, 230b, and 230c may each output a second detection signal. Each of the first detection signal and each of the corresponding second detection signal may be assembled in a signal assembly unit 331. For example, the signals from the pair of sensor units may be assembled into one signal assembly unit 331. Therefore, signals from three pairs of paired sensor units may each be assembled into three signal assembly units 331, each receiving one pair of signals. The signals assembled in the signal assembly unit 331 may be converted into visible data in the signal converter 332.

For the converted data, noise may be adjusted in the signal analysis unit 333. The signals output from three pairs of sensor unit pairs may be simultaneously adjusted for increased noise-reduction precision. Accordingly, the electronic device can output a refined signal including the user motion with precision.

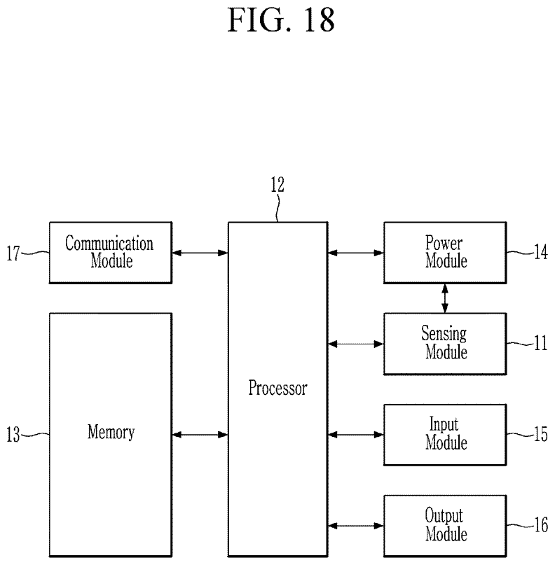

FIG. 18 is a schematic block diagram of an electronic device according to an embodiment. Referring to FIG. 18, the electronic device may further include a module or a device having additional capabilities in addition to the optical sensor component, the signal processor, and the battery. An electronic device 10 according to an embodiment may include a sensing module 11, a processor 12, a memory 13, a power module 14, an input module 15, an output module 16, and a communication module 17. The sensing module 11 may correspond to the light emitter of the optical sensor component.

The processor 12 may include a central processing unit (CPU), an application processor (AP), etc. In some cases, processor 12 is an intelligent hardware device, (e.g., a general-purpose processing component, a digital signal processor (DSP), a central processing unit (CPU), a graphics processing unit (GPU), a microcontroller, an application-specific integrated circuit (ASIC), a field programmable gate array (FPGA), a programmable logic device, a discrete gate or transistor logic component, a discrete hardware component, or a combination thereof. In some cases, processor 12 is configured to operate a memory array using a memory controller. In other cases, a memory controller is integrated into the processor 12. In some cases, the processor 12 is configured to execute computer-readable instructions stored in a memory to perform various functions. In some embodiments, the processor 12 includes special-purpose components for modem processing, baseband processing, digital signal processing, or transmission processing.

The memory 13 may store data information for the operation of the processor 12 or the sensing module 11. When the processor 12 executes an application stored in the memory 13, input control signals, or other signals may be transmitted to the sensing module 11, or other components, and the sensing module 11 may sense light to process the provided signals.

The power module 14 may include a power supply module, such as a power adapter or a battery device, and a power module that converts a power supplied by the power supply module to generate power required for the operation of the electronic device 10. For example, the power module 14 may correspond to the battery 400 described above with reference to FIG. 2 and FIG. 11.

The electronic device 10 may further include an input module 15, an output module 16 (e.g., a non-image output module), a communication module 17, or other additional components.

The input module 15 may provide input information to the processor 12 or the sensing module 11. The input module 15 may include various sensor modules as well as physical buttons. Examples of sensor modules may include biometric sensors such as blood pressure sensors, electrocardiogram sensors, and heart rate sensors as well as distance sensors, pressure sensors, position sensors, touch sensors, motion recognition sensors, digitizers, optical sensors, photoelectric conversion sensors, temperature sensors, etc.

The output module 16 may receive information other than the image received from the processor 12 and provide the information to the user. The output module 16 may include, for example, a light emitting module.

The communication module 17 is a module responsible for transmitting/receiving information between the electronic device 10 and an external device, and may include a receiver and a transmitter. The communication module 17 may include various wireless modules such as a mobile communication module, a Wi-Fi module, and a Bluetooth module.

At least one of the components of the electronic device 10 described above may be included in the optical sensor component and the signal processor according to the embodiments described above.

While the present disclosure has been described in connection with the exemplary embodiments, it is to be understood that the disclosure is not limited to the disclosed embodiments, but, on the contrary, is intended to cover various modifications and equivalent arrangements included within the spirit and scope of the appended claims.

What is claimed is:

1. An electronic device, comprising:
a substrate;
a first sensor assembly disposed on the substrate, wherein the first sensor assembly includes a light emitter and a first optical sensor adjacent to the light emitter, and wherein the first sensor assembly is configured to output a first detection signal;
a second sensor assembly including a second optical sensor spaced apart from the first sensor assembly and disposed on the substrate, wherein a distance between the second optical sensor and the light emitter is greater than a distance between the second optical sensor and the first optical sensor, and wherein the second sensor assembly is configured to output a second detection signal; and
a signal processor configured to receive the first detection signal and the second detection signal, and to generate a refined detection signal based on the first detection signal and the second detection signal.

2. The electronic device of claim 1, wherein:
the second sensor assembly does not include a light emitter.

3. The electronic device of claim 1, further comprising:
a first conductive line disposed on the substrate, wherein the first conductive line connects the first sensor assembly and the signal processor, and the first conductive line connects the second sensor assembly and the signal processor;
a first insulation layer disposed on the first conductive line, wherein the first insulation layer includes a connection hole; and
a first electrode that overlaps the connection hole and connects the first sensor assembly and the first conductive line, and connects the second sensor assembly and the first conductive line.

4. The electronic device of claim 3, wherein:
the first optical sensor completely surrounds the light emitter.

5. The electronic device of claim 1, further comprising:
a first insulation layer disposed on the substrate; and
a second conductive line disposed on the first insulation layer and connecting the first sensor assembly and the signal processor, and connecting the second sensor assembly and the signal processor.

6. The electronic device of claim 5, wherein:
the first optical sensor partially surrounds the light emitter.

7. The electronic device of claim 1, wherein:
the first optical sensor includes a first part and a second part, wherein the first part and the second part are disposed on opposite sides of the light emitter, and are spaced apart from each other, and
the second optical sensor includes a third part and a fourth part, wherein the first part is disposed between the light emitter and the third part, and the second part is disposed between the light emitter and the fourth part.

8. The electronic device of claim 1, wherein:
the first sensor assembly and the second sensor assembly comprise a plurality of assemblies.

9. The electronic device of claim 8, further comprising:
a first conductive line disposed on the substrate and connecting the first sensor assembly and the signal processor;
a first insulation layer including a connection hole disposed on the first conductive line;
a first electrode overlapping the connection hole; and
a second conductive line disposed on the first insulation layer and connecting the second sensor assembly and the signal processor.

10. The electronic device of claim 8, wherein
the first sensor assembly and the second sensor assembly are alternately disposed.

11. The electronic device of claim 1, further comprising:
a second insulation layer disposed on the substrate, wherein the second insulation layer includes a first opening, a second opening, and a third opening,
wherein the first optical sensor overlaps the first opening, the light emitter overlaps the second opening, and the second optical sensor overlaps the third opening.

12. The electronic device of claim 11, wherein:
the second insulation layer includes a light blocking material.

13. The electronic device of claim 1, wherein:
the light emitter includes an organic light emitting diode.

14. The electronic device of claim 1, wherein:
the light emitter includes a first sub-light emitter configured to emit a light having a first wavelength and a second sub-light emitter configured to emit a light having a second wavelength different from the first wavelength.

15. The electronic device of claim 1, further comprising:
a second electrode covering the light emitter, the first optical sensor, and the second optical sensor.

16. A wearable electronic device, comprising:
a housing including an inner cover defining an inner surface of a wearable electronic device;
an electronic part disposed in the housing,
wherein the electronic part includes:
a substrate;
an optical sensor component including a first sensor assembly and a second sensor assembly disposed on the substrate,
wherein the first sensor assembly includes a light emitter and a first optical sensor adjacent to the light emitter, and configured to output a first detection signal,
wherein the second sensor assembly includes a second optical sensor spaced apart from the first sensor assembly, and configured to output a second detection signal, and
wherein a distance between the second optical sensor and the light emitter is greater than a distance between the second optical sensor and the first optical sensor; and
a signal processor configured to receive the first detection signal and the second detection signal, and to generate a refined detection signal based on the first detection signal and the second detection signal.

17. The wearable electronic device of claim 16, wherein:
the light emitter emits light toward the inner surface.

18. The wearable electronic device of claim 16, wherein:
a first end and a second end of the optical sensor component are connected to a first end and a second end of the signal processor, respectively.

19. The wearable electronic device of claim 16, wherein:
the optical sensor component includes a plurality of first sensor assembly and a plurality of second sensor assembly, wherein each of the plurality of first sensor assembly and each of the plurality of second sensor assembly are alternately arranged.

20. The wearable electronic device of claim 16, wherein:
the housing has a ring shape.
